(19) United States
(12) Patent Application Publication
(10) Pub. No.: US 2022/0387701 A1
(43) Pub. Date: Dec. 8, 2022

(54) INFUSION SYSTEM, ROTOR MODULE FOR USE IN SUCH AN INFUSION SYSTEM AND METHOD FOR DETERMINING A FLOW RATE OF AN INFUSION FLUID IN SUCH AN INFUSION SYSTEM

(71) Applicant: B. Braun Melsungen AG, Melsungen (DE)

(72) Inventor: Michael Fiege, Kassel (DE)

(21) Appl. No.: 17/773,318

(22) PCT Filed: Nov. 4, 2020
(86) PCT No.: PCT/EP2020/080881

§ 371 Date: Apr. 29, 2022

(30) Foreign Application Priority Data
Nov. 8, 2019 (DE) 10 2019 217 315.2

(51) Int. Cl. A61M 5/14

(52) U.S. Cl. CPC A61M 5/14; A61M 5/1411; A61M 5/1424

(57) ABSTRACT

An infusion system includes a chamber for accommodating an infusion fluid and an infusion line for transferring the infusion fluid from the chamber. The infusion line includes at least a rotor module or is connected to a rotor module at least at a downstream end of the infusion line. The rotor module includes a rotor that is drivable by the infusion fluid.

### CROSS-REFERENCES TO RELATED APPLICATIONS

This application is the United States national stage entry of International Application No. PCT/EP2020/080881, filed Nov. 4, 2020, and claims priority to German Application No. 10 2019 217 315.2, filed Nov. 8, 2019. The contents of International Application No. PCT/EP2020/080881 and German Application No. 10 2019 217 315.2 are incorporated by reference herein in their entireties.

### FIELD

The invention relates to an infusion system, a rotor module for use in such infusion system, a method for determining a flow rate of an infusion fluid in such an infusion system as well as a respective computer-implemented method and a corresponding computer program product.

### BACKGROUND

Electric pump systems with an input interface are widely used for a precise control of a flow rate. In particular for mobile purposes, electric pump systems are, however, not applicable, and are also usually only used for complex infusion regimes due to the comparatively high acquisition costs.

For infusions that are not controlled by electric pump systems, in which a flow rate can be directly set at a display, it is up to the user to estimate the flow rate indirectly, for example on the basis of the drop rate in a drip chamber. In such event, misjudgments may occur due to subjective perception effects. In the event of observing the drop rate in a drip chamber for a supposed determination of the flow rate, this is also due to the fact that the size of the drops depends on the drop rate, the temperature of the infusion solution, the design of the drip edge, the surface tension of the infusion solution, the density of the infusion solution and the ambient pressure. Further, particularly in the case of pressure infusions, a drop rate may no longer be signaled in the drip chamber, since the flow rate is that high that no drops form at all or are at least no longer recognizable as such and only a continuous flow of fluid may be observed, which does not permit any estimation of the flow rate.

Another known alternative to electric pump systems are among others elastomeric pumps, in which the pump pressure is built up via the elongation of an elastomeric body, wherein the infusion rate is specified by a flow limiter, such as a fine capillary. Such elastomeric pumps are usually configured to apply an infusion flow rate theoretically fixed predetermined by the design and layout of the system. Nevertheless, in practice, commonly used elastomeric pumps exhibit flow rate variations with a relatively high tolerance range, since the flow rate may alter due to changes in tension on the elastic fluid reservoir. Other influencing factors on the flow rate of elastomeric pumps are, for example, the temperature, the filling quantity, the viscosity of the infusion fluid and the ambient pressure. The user has no way of detecting or influencing a respectively resulting deviation from the theoretically fixed predetermined flow rate. In the absence of corresponding regulation opportunities, a tolerable flow rate may only be ensured under strictly predefined boundary conditions.

### SUMMARY

In consideration of the disadvantages associated with the prior art, it is an object of the present invention to provide an infusion system and method for use of an infusion fluid in such an infusion system, which allows a simple determination of a flow rate.

According to the invention, the infusion system comprises a chamber for accommodating an infusion fluid, and an infusion line for transferring the infusion fluid from the chamber, wherein the infusion line comprises at least one rotor module or is connected to a rotor module at least at a downstream end of the infusion line, wherein the rotor of the rotor module is drivable by the infusion fluid.

The rotational speed of the rotor corresponds to the flow rate of the infusion fluid due to the arrangement of the rotor in the flow path of the infusion fluid. The rotor is configured to be finely rotated, particularly for lower flow rates, to be drivable by a rotational speed corresponding to the flow rate. The flow rate of the infusion fluid may be objectively determined based on the rotational speed of the rotor in conjunction with the inlet cross section.

Provided that the rotor module is not comprised by the infusion line but is connected to a downstream end of the infusion line, such connection may be mediate or direct. If a change in the flow rate of the infusion fluid is possible along the connection path, for example by interposition of further members, a mediate or indirect connection may be particularly advantageous, so as to determine the actual flow rate at a location relevant for the dosing of the infusion. At the upstream side and/or downstream side of the rotor, regardless of whether the rotor module in integrated into an infusion line or whether the rotor module in connected to the end of a (possibly “first”) infusion line, further members of the infusion system may be attached or integrated, possibly a further (possibly then “second”) infusion line and/or further devices, some of which are particularly described below.

Particularly, the rotor is at least in certain areas visible from the outside via a transparent housing portion of the rotor module.

This allows the rotational movement of the rotor to be detected and evaluated by an optical detection unit described later. Alternatively or in addition, a mark of the rotor described below, which is observable in the transparent housing portion, may be used to determine the flow rate. Further alternatively or in addition, the rotational speed of the rotor and therefore the flow rate corresponding thereto may be indicated by an indicator independent of the transparent housing portion, such as a scale, which is, for example, connected to the rotor via a transmission. The rotor module may alternatively or in addition generate a signal corresponding to the rotational speed of the rotor, which may be transferred to a display independent from the transparent housing portion or an external system. By the transfer to an external system, the flow rates may be recorded and/or warning notifications may be issued if predefined thresholds are exceeded. The latter is particularly advantageous if it may not be ensured that the infusion recipient as such is able to react appropriately, such that respective support staff is informed.

According to a further development, the rotor comprises at least one mark observable via the transparent housing portion at least during its passing of the transparent housing portion.

Such a mark is easily visually perceptible and/or detectable and/or, depending on the rotational speed and design, may generate a pattern corresponding to a predetermined rotational speed or a predetermined rotational speed range. With regard to the latter, an insufficient or excessive flow rate may be concluded directly in the event of an absence of forming the pattern.

According to an embodiment, the chamber is a drip chamber of a gravity system or of a pressure infusion system.

Thus, it is also possible to objectively determine the flow rate of the infusion fluid by the rotor module for gravity or also for pressure infusion pumps. In particular, however, the subjective perception with respect to the drop rate observed through the drip chamber and the flow rate derived from this may also be compared with the objectively determined flow rate for control or training purposes.

According to an alternative embodiment, the chamber is an infusion reservoir of an elastomeric pump.

Thus, it is possible to extend the field of application of elastomeric pumps to environments, in which a change in the flow rate beyond predetermined tolerances is possible, since it is possible to react due to an inadmissible deviation becoming known.

According to a further development of the present invention, the infusion line comprises a flow rate reducer, in particular a roller clamp between the infusion reservoir and the rotor module.

Hence, the user may not only react to inadmissible deviations of the drop rate by selectively influencing the environment or other boundary conditions, but may directly adjust the flow rate. For this purpose, a respective flow rate reducer may be set such that it already reduces the flow rate to a predetermined value compared to a maximum flow rate during normal operation, so that in the event of an inadmissible deviation, not only a reduction but also an increase in the flow rate may be executed by the flow rate reducer depending on the required correction direction. This is implementable in a simple manner via a roller clamp, with which respective users are also well acquainted. This is particularly advantageous for elastomeric pumps, which otherwise do not provide an opportunity to adjust the flow rate.

Particularly, the infusion line and/or the rotor module comprises a filter, and the rotor is arranged downstream of the filter in a downstream direction.

Due to the filter, particles may be filtered out of the infusion fluid. Since the flow behavior of the infusion fluid may change after filtering of the particles, the flow rate is determined via the rotational speed of the rotor only in the flow path of the filtered infusion fluid.

According to a further development, the infusion system comprises an optical detection unit, by which the rotational frequency of the rotor is detectable, in particular is convertible into a flow rate of the infusion fluid and displayable and/or storable.

The optical detection unit may be configured as scanner or camera. Particularly, the optical detection unit is a mobile device, such as a smartphone or a tablet (tablet computer), wherein the smartphone or the tablet comprises a respectively integrated scanner or a respectively integrated camera. The conversion of the detected rotational speed may be carried out by a calculation algorithm, for example by considering the inlet diameter of the infusion line, or by comparison with stored tabular values. In addition to displaying and/or storing by the optical detection unit, the latter may also be configured to transfer signals to a superordinate system for converting the rotational speed into a flow rate, for displaying and/or storing the flow rate.

Further, the invention is directed to a rotor module for use in an infusion system described above, wherein the rotor module comprises a connection for being connected to an infusion line, and the rotor module comprises a rotor, which is arranged such that the infusion fluid is routable via the rotor to a downstream outlet of the rotor module.

Hence, the rotor module may be connected to a conventional infusion system to determine the flow rate. This means that the rotor module may be retrofitted. In addition, the rotor module may be used on demand.

The invention is also directed to a method for determining a flow rate of an infusion fluid in an infusion system described above, comprising the steps of:
acquiring a rotational frequency of the rotor of the rotor module, and
converting the acquired rotational frequency of the rotor into a flow rate of the infusion fluid.

The conversion is based on a correlation between the rotational frequency of the rotor and the flow rate of the infusion fluid. The advantages of such procedure are analogous to those described for the infusion system according to the invention.

According to an embodiment of the method, the acquisition of the rotational frequency of the rotor is carried out via an optical detection unit, preferably a scanner or a camera.

For this purpose, the optical detection unit is oriented to the rotor. If the optical detection unit is no part of the rotor module, at least one housing portion transparent to the optical detection unit is provided, via which the rotational frequency of the rotor can be detected optically. The acquisition of the rotational frequency by the optical detection unit is particularly carried out over a predetermined period of time in order to avoid subjecting any short-term fluctuations in rotational speed to a snapshot and/or if the determination of the rotational speed is based on the change in an acquisition state.

Particularly, the optical detection unit acquires the rotational frequency of the rotor via a mark provided on the rotor.

Here, for example, the change in position of the mark over a predetermined period of time or a pattern forming over the mark in conjunction with the rotational frequency of the rotor may be detected by the optical detection unit.

According to a further development, die optical detection unit displays the flow rate converted from the rotational frequency of the rotor.

The display may also provide different color background, for example, to visualize critical flow rates in red. If critical flow rates are detected, the optical detection unit, such as a smartphone or tablet, may also be configured to, alternatively or in addition, emit an acoustic, optical and/or haptic signal. Also alternatively or in addition, the optical detection unit may transmit respective warning notifications and/or messages to external systems.

According to an embodiment, the optical detection unit is mobile and optionally displays operating instructions for the acquisition process, wherein the mobile optical detection unit is in particular integrated into a smartphone or a tablet computer.

A mobile optical detection unit, such as the smartphone or tablet mentioned above with the respectively integrated camera or scanner, enables flexible use of the optical detection unit. In particular, smartphones or tablets are already carried along by users as standard anyway, so that they may be upgraded in a simple manner by uploading a computer program product described later.

To facilitate the process of acquiring the rotational frequency of the rotor and other method steps, the optical detection unit may also directly display operating instructions so that, in particular, skilled personnel with no medical training may directly implement the method according to the invention. For example, the optical detection unit may indicate that its orientation to the rotor has not been performed correctly, that it must be held still, that the acquisition process has ended, or that the acquisition process must be repeated.

The invention is also directed to a computer-implemented method for determining a flow rate of an infusion fluid in an infusion system described above, comprising the steps of:
triggering an acquisition of a rotational frequency of the rotor of the rotor module,
converting the acquired rotational frequency of the rotor into a flow rate of the infusion fluid, and
displaying and/or storing the flow rate of the infusion fluid converted from the rotational frequency of the rotor and/or transferring it to an external display device and/or storage device.

The advantages here are analogous to those described above for the method. The triggering of the detection may be done manually via an input interface or may be provided in an automated system continuously, at predetermined times or event-dependent. A triggering event may be, for example, a change in ambient temperature, which, as mentioned at the beginning, may affect the flow rate of the infusion fluid.

A further subject matter of the present invention relates to a computer program product comprising commands, which when executing the program cause it to execute the computer-implemented method described above.

Such a computer program product may be made available, for example, in the form of an app, i.e., a user program, on a smartphone or a tablet.

Moreover, the computer program product may comprise further program sequences, for example to query the technical requirements of an optical detection unit for sufficient reliability of the acquisition of the rotational frequency of the rotor on the basis of a test sequence and to prevent an acquisition if this is not ensured. Likewise, access restrictions to the method or to parts thereof may be implemented via the computer program product.

### BRIEF DESCRIPTION OF THE DRAWING FIGURES

Features, functionalities and advantages of the invention are also described below with reference to the drawings by way of exemplary embodiments.

FIG. 1 shows a schematic view of an infusion system according to a first exemplary embodiment; and

FIG. 2 shows a schematic view of an infusion system according to a second exemplary embodiment.

### DETAILED DESCRIPTION

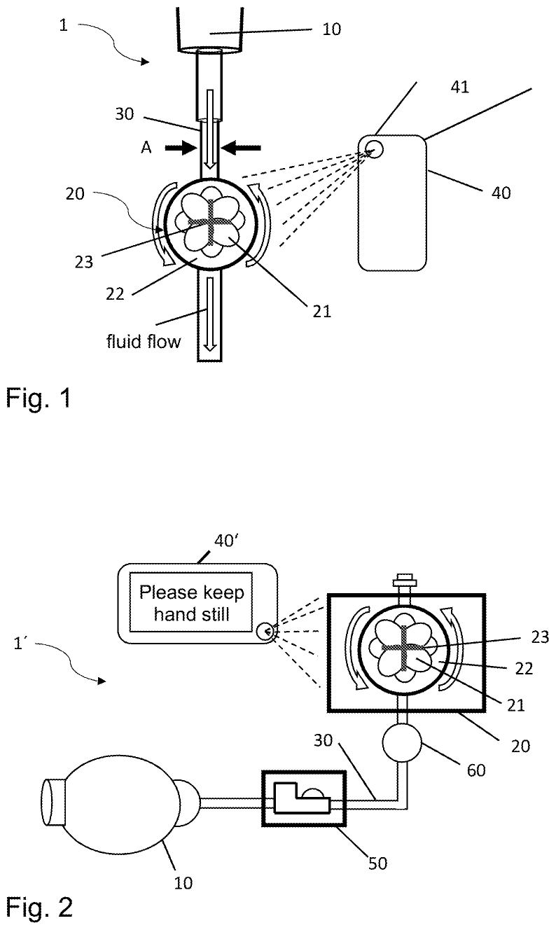

The schematic view of an infusion system 1 according to a first exemplary embodiment represented in in FIG. 1 shows a chamber 10, here, a drip chamber 10 of a gravity or pressure infusion system. In the flow direction of the infusion fluid indicated by the arrows, a rotor module 20 with a rotor 21 is subsequent to the infusion line 30. Here, the rotor module 21 is comprised by the infusion line 30, but may also be arranged downstream of the infusion line 30 in the flow direction of the infusion fluid and connected thereto and in turn comprises a further line or a connection to further components on a fluid outlet side, i.e. on the side on which an outlet is provided for the infusion fluid flowed through the rotor module. The rotor module 20 also need not be directly connected to the infusion line adjoining the drip chamber, but may also form such a connection indirectly via components arranged therebetween.

The rotor module 20 comprises a transparent housing portion 22 on a side that permits a view of the rotational axis of the rotor 21 and/or of a portion of the rotor 21 extending radially with respect to the rotational axis of the rotor 21, that is, a portion in a plane perpendicular to the rotational axis of the rotor 21. In the embodiment shown, both the rotor 21 perpendicular to the rotational axis and the mark 23 are optically detectable via the transparent housing portion 22. The mark 23 comprises two perpendicular marking lines that intersect in the rotational axis. The mark is detected by the optical detection unit 40, here configured as a scanner, and hereby the rotational speed of the rotor is determined. For example, the marking lines form a virtual line pattern as a function of the rotational speed, i.e. a line pattern deviating from the actual marking lines in a resting state, wherein the rotational speed may be derived by the distance between the virtual lines. Alternatively, the change in a position of the mark 23 or a marking line of the mark 23 may be used to determine the rotational speed. Since the inlet cross section A is known, the optical detection unit 41 may calculate the current flow rate of the infusion fluid from the rotational speed of the rotor 21. For this purpose, the optical detection unit may also comprise an input unit, via which predetermined inlet cross sections A may be selected and/or input, provided that variants may occur. The flow rate converted from the rotational speed is subsequently displayed in the display 41 of the optical detection unit 40. The optical detection unit 40 may also transmit the determined rotational speed and/or the converted flow rate to external display devices and/or storage devices.

FIG. 2 shows a schematic view of an infusion system 1′ according to a second exemplary embodiment of the invention, in which the same reference signs designate the same or corresponding elements. In this second embodiment, the chamber 10′ is an infusion reservoir of an elastomeric pump. Again, an infusion line 30, which comprises a flow rate reducer 50, a filter 60, and a rotor module 20, follows in the flow direction of the infusion fluid from the chamber 10′. The determination of the rotational speed of the rotor 21 is carried out analogously to the procedure described for the first embodiment.

The flow rate reducer 50, which is here configured as a roller clamp, may change the flow rate of the infusion fluid. For example, if an inadmissible deviation of the flow rate is detected via the rotational speed of the rotor 21, the flow rate may be adjusted to a predetermined target value or an admissible range of the flow rate. Accordingly, the flow rate reducer in particularly arranged upstream of the rotor module 20 in order to allow to check the result of the adjustment.

The filter 60 is provided in the detection line to filter potential particles from the infusion fluid. Accordingly, the rotor module is arranged downstream of the filter 60 in order to determine the result the rotational speed and therefore the flow rates, without the influence by potential particles.

Alternatively to the first optical detection unit 40 in FIG. 1 for the first embodiment, FIG. 2 shows an optical detection unit 40′, which is here configured as a smartphone. As indicated by the dashed lines, the rotational speed of the rotor 21 may be detected via the camera lens of the smartphone. For this purpose, a scanner function of the camera or an image processing program, which, for example, evaluates a video sequence is used. At the same time, the display of the smartphone or the optical detection unit 40′, respectively, shows an operating instruction, via which the user is instructed to hold the smartphone still during the detection process. For this purpose, it may be provided that the display indicates whether the optical detection unit 40′ is held sufficiently still by means of visual, acoustic or haptic signs. For example, the display may have a green background when the smartphone is held still, while increasing movement results in a discoloration from green to red until, for example, the measurement is terminated or discarded at a movement threshold. In addition to optical indications, however, an acoustic signal or an acoustic signal sequence and/or haptic signals, such as a vibration corresponding to a movement of the optical detection unit 40′, may also be used alternatively or in addition.

The invention is not limited to the described embodiments. Also, details given to various embodiments are in principle transferable to other embodiments, provided that they are not mutually exclusive. Even if a scanner has been described for the first and second embodiments, this does not have to be designed as a pure scanner unit, but may also be provided via a smartphone, as in FIG. 2, or a tablet. Accordingly, the scanner may also be adapted by a camera or a camera may be used with an image processing program that is not necessarily limited to scanner-adapting acquisition methods. Smartphones and tablets with application programs installed on them are a familiar medium for users. Thus, in the embodiment shown in FIG. 2, a tablet (computer) may be used instead of a smartphone. In addition, a process sequence guided by the application program may reduce errors for inexperienced users and generally simplify the process flow.

1. An infusion system comprising:
a chamber for accommodating an infusion fluid; and
an infusion line for transferring the infusion fluid from the chamber,
the infusion line comprising or connected to a rotor module at least at a downstream end of the infusion line,
the rotor module comprising a rotor that is drivable by the infusion fluid, and
the chamber comprising an infusion reservoir of an elastomeric pump.

2. The infusion system according to claim 1, wherein the rotor is at least in certain areas visible from the outside via a transparent housing portion of the rotor module.

3. The infusion system according to claim 2, wherein the rotor comprises at least one mark observable via the transparent housing portion at least during its passing of the transparent housing portion.

4. (canceled)

5. (canceled)

6. The infusion system according to claim 1, wherein the infusion line comprises a flow rate reducer between the infusion reservoir and the rotor module.

7. The infusion system according to claim 1, wherein the infusion line and/or the rotor module comprises a filter, and the rotor is arranged downstream of the filter in a downstream direction.

8. The infusion system according to claim 1, wherein the infusion system comprises an optical detection unit, by which the rotational frequency of the rotor is detectable.

9. A rotor module for use in the infusion system according to claim 1, wherein the rotor module comprises a connection for being connected to an infusion line, and the rotor module comprises a rotor which is arranged such that the infusion fluid is routable via the rotor to a downstream outlet of the rotor module.

10. A method for determining a flow rate of the infusion fluid in the infusion system according to claim 1, comprising the steps of:
acquiring a rotational frequency of the rotor; and
converting the rotational frequency into a flow rate of the infusion fluid.

11. The method according to claim 10, wherein acquisition of the rotational frequency of the rotor is carried out via an optical detection unit.

12. The method according to claim 11, wherein the optical detection unit acquires the rotational frequency of the rotor via a mark provided on the rotor.

13. The method according to claim 11, wherein the optical detection unit displays the flow rate.

14. The method according to claim 11, wherein the optical detection unit is mobile.

15. A computer-implemented method for determining a flow rate of an infusion fluid in an infusion system according to claim 1, comprising the steps of:
triggering an acquisition of a rotational frequency of the rotor;
converting the rotational frequency of the rotor into a flow rate of the infusion fluid; and
displaying and/or storing the flow rate of the infusion fluid and/or transferring the flow rate of the infusion fluid to an external display device and/or storage device.

16. A computer program product comprising commands, which when executed cause it to execute the computer-implemented method according to claim 15.
